(19) United States
(12) Patent Application Publication
(10) Pub. No.: US 2026/0315748 A1
(43) Pub. Date: Oct. 15, 2026

(54) DISTAL STABILIZER AND IMAGING METHOD FOR DISTAL STABILIZER

(71) Applicant: Bolt Medical Inc., Tokyo (JP)

(72) Inventor: Naoki INUZUKA, Tokyo (JP)

(73) Assignee: Bolt Medical Inc., Tokyo (JP)

(21) Appl. No.: 19/484,290

(22) PCT Filed: May 7, 2024
(86) PCT No.: PCT/JP2024/016939

§ 371 Date: Nov. 13, 2025

(51) Int. Cl. A61M 25/04; A61M 25/00

(52) U.S. Cl. CPC A61M 25/04; A61M 2025/0004; A61M 2205/32

(57) ABSTRACT

The present invention provides: a distal stabilizer that makes it possible to easily ascertain an enlarged-diameter state of an anchor device; and an imaging method for the distal stabilizer. A distal stabilizer 1 is used for catheter delivery in a biological lumen. The distal stabilizer 1 comprises: a delivery wire 3; and a locking stent 2 extending from the distal end of the delivery wire 3 and including a cylindrical part 11 which is locked to the inner wall of the biological lumen. At least a portion of the cylindrical part 11 includes a visible part that is impermeable to X-rays.

### TECHNICAL FIELD

The present disclosure relates to a distal stabilizer, and an imaging method for a distal stabilizer.

### BACKGROUND ART

In a biological lumen such as an artery of a patient, a treatment using a treatment device or a treatment using a catheter itself as a treatment device is performed by delivering the treatment device to a target position using a passage within the catheter whose distal end is guided to the vicinity of the target position. For example, Patent Document 1 discloses an anchor device including a cylindrical portion at a distal end of a delivery wire. When the cylindrical portion is released from a microcatheter and allowed to expand, since the cylindrical portion is locked (anchored) to the inner wall of the biological lumen, the operation of delivering the treatment catheter that is placed over the microcatheter to the vicinity of the target position can be easily performed.

### CITATION LIST

### Patent Document

### Patent Document 1: U.S. Pat. No. 9,682,216

### DISCLOSURE OF THE INVENTION

### Problems to be Solved by the Invention

The anchor device performs locking in contact with the inner wall of the biological lumen while pressing the inner wall through self-expansion (diametrical expansion) within the biological lumen. Accordingly, in a situation where the anchor device has completely expanded in diameter, for example, the pressing force acting on the inner wall of the biological lumen is low, or completely lost, and thus the anchor device is not able to perform sufficient locking. Consequently, it is desired that the anchor device is used under the condition that the anchor device contacts the inner wall of the biological lumen while being not in a full but midway expansion in diameter.

However, it has not been considered for a conventional anchor device to grasp the diametrically expanded state.

An object of the present disclosure is to provide a distal stabilizer that allows the diametrically expanded state of an anchor device to be easily grasped, and an imaging method for the distal stabilizer.

### Means for Solving the Problems

The present disclosure achieves this object by such solutions as described below. It should be noted that to facilitate understanding, a description is provided by attaching reference symbols corresponding to the embodiments of the present disclosure; however, it is not to be limited thereto.

A first aspect of the present invention relates to a distal stabilizer (1) designed for catheter delivery within a biological lumen. The distal stabilizer includes: a linear delivery member (3) ; and an anchor device (2, 2B, 2C, 2D, 2E, 2F) including a cylindrical portion (11) that extends from a distal end of the linear delivery member (3) and is locked to an inner wall of the biological lumen. The cylindrical portion (11) has a structure in which a plurality of cells (20), each of which has a shape surrounded by wire-shaped members (22a, 22b, 22c, 22d), are arranged. And at least a portion of an area in which the cells (20) of the cylindrical portion (11) are provided includes a radiographically visible part (2, 101, 102, 103, 104, 105, 106, 107, 108, 109).

A second aspect of the present invention relates to the distal stabilizer (1) as described in the first aspect, in which the radiographically visible part (2, 101, 102, 103, 104, 105, 106, 107, 108, 109) is provided at a part of the cylindrical portion (11) to make a diametrically expanded state thereof visible.

A third aspect of the present invention relates to the distal stabilizer (1) as described in the first aspect or the second aspect, in which at least one of the cells (20) includes an open cell portion having at least one free convex end (23), and the radiographically visible part (2, 101, 102, 103, 104, 105, 106, 107, 108, 109) is provided to at least a wire-shaped member (22a, 22b) forming the free convex end (23).

A fourth aspect of the present invention relates to an imaging method for acquiring an image of the distal stabilizer (1) as described in any one of the first aspect to third aspect within a biological lumen. The method includes: a first step of imaging the radiographically visible part (2, 101, 102, 103, 104, 105, 106, 107, 108, 109) of the distal stabilizer (1) delivered together with the linear delivery member (3) into the biological lumen; and a second step of imaging the radiographically visible part (101, 102, 103, 104, 105, 106, 107, 108, 109) after moving the cylindrical portion (11) to a position further on a distal side of the biological lumen than a position of the cylindrical portion (11) delivered in the first step.

A fifth aspect of the present invention relates to an imaging method for acquiring an image of the distal stabilizer (1) as described in any one of the first aspect to third aspect within a biological lumen. The method includes: a first step of imaging the radiographically visible part (2, 101, 102, 103, 104, 105, 106, 107, 108, 109) of the distal stabilizer (1) delivered together with the linear delivery member (3) into the biological lumen, prior to an operation of drawing the linear delivery member (3) ; and a second step of imaging the radiographically visible part (2, 101, 102, 103, 104, 105, 106, 107, 108, 109) after or during the operation of drawing the linear delivery member (3).

A sixth aspect of the present invention relates to an imaging method for acquiring an image of the distal stabilizer (1) as described in any one of the first aspect to third aspect within a biological lumen. The method includes: a first step of imaging the radiographically visible part (2, 101, 102, 103, 104, 105, 106, 107, 108, 109) of the distal stabilizer (1) delivered together with the linear delivery member (3) into the biological lumen; and a second step of imaging the radiographically visible part (2, 101, 102, 103, 104, 105, 106, 107, 108, 109) after the cylindrical portion (11) has shifted from a position thereof within the biological lumen at a time of imaging in the first step.

(Catheter delivery method 1) A delivery method of a catheter within a biological lumen using the distal stabilizer (1) as described in any one of the first aspect to the third aspect may be performed. The method includes steps described below. In a first step: delivering the first catheter (5) by inserting the first catheter (5) into the second catheter (6) having a larger inner diameter than the first catheter (5) ; pushing the first catheter (5) out from a distal end of the second catheter (6), followed by arranging the distal end of the first catheter (5) in a vicinity of a target position. In a second step: housing the distal stabilizer (1) in a diametrically reduced state into the first catheter (5). In a third step: releasing the housed distal stabilizer (1) from the distal end of the first catheter (5) and locking the housed distal stabilizer (1) to an inner wall. In a fourth step: imaging a radiographically visible part (2, 101, 102, 103, 104, 105, 106, 107, 108, 109) in a state placing the second catheter 6 over the linear delivery member (3) of the distal stabilizer (1) and a state locking the distal stabilizer (1) to the inner wall. In a fifth step: advancing the catheter including the second catheter (6) toward the distal side while continuing the fourth step, in a state locking the distal stabilizer (1) to the inner wall. And in a sixth step: reducing the diameter of the distal stabilizer (1) and resheathing the distal stabilizer (1) into either one of the catheters (5, 6), and drawing out the distal stabilizer (1) from the biological lumen after the fifth step.

(Catheter delivery method 2) In addition, in (Catheter delivery method 1), the fifth step may be performed in a state placing the second catheter (6) over the first catheter (5).

(Catheter delivery method 3) In addition, in (Catheter delivery method 1), the fifth step may be performed in a state drawing out the first catheter (5).

(Treatment method) In addition, after the sixth step of the above-mentioned (Catheter delivery method 1) to (Catheter delivery method 3), a treatment method may be performed with a therapeutic action by inserting the treatment device (7) into the second catheter (6), and bringing the treatment device (7) out from the distal end of the second catheter (6).

### Effects of the Invention

According to the present disclosure, it is possible to provide a distal stabilizer that allows an easy grasp for the diametrically expanded state of the anchor device, and an imaging method for the distal stabilizer.

### BRIEF DESCRIPTION OF THE DRAWINGS

FIG. 1 is a view showing a delivery system 10 including a distal stabilizer 1 according to a first embodiment;

FIG. 2 is a drawing viewing a diametrically expanded state of a locking stent 2 from a direction orthogonal to an axial direction LD;

FIG. 3 is a drawing viewing the locking stent 2 from a proximal LD1 side;

FIG. 4 is an expanded view showing the locking stent 2 expanded onto a plane virtually;

FIG. 5 is an enlarged view of a region A indicated in FIG. 4; FIG. 6 is a schematic diagram showing an example of a procedure performed with the delivery system 10 including the distal stabilizer 1;

FIG. 7 is a schematic diagram showing an example of a procedure performed with the delivery system 10 including the distal stabilizer 1;

FIG. 8 is a schematic diagram showing an example of a procedure performed with the delivery system 10 including the distal stabilizer 1;

FIG. 9 is a schematic diagram showing an example of a procedure performed with the delivery system 10 including the distal stabilizer 1;

FIG. 10 is a schematic diagram showing an example of a procedure performed with the delivery system 10 including the distal stabilizer 1;

FIG. 11 is a schematic diagram showing an example of a procedure performed with the delivery system 10 including the distal stabilizer 1;

FIG. 12 is a schematic diagram showing an example of a procedure performed with the delivery system 10 including the distal stabilizer 1;

FIG. 13 is a schematic diagram showing an example of a procedure performed with the delivery system 10 including the distal stabilizer 1;

FIG. 14 is a schematic diagram showing an example of a procedure performed with the delivery system 10 including the distal stabilizer 1;

FIG. 15 is a view showing an imaging result acquired in a first step of a first imaging method;

FIG. 16 is a view showing an imaging result acquired in a second step of the first imaging method;

FIG. 17 is a view showing an imaging result acquired in a first step of a second imaging method;

FIG. 18 is a view showing an imaging result acquired in a second step of the second imaging method;

FIG. 19 is a view showing an imaging result acquired in a first step of a third imaging method;

FIG. 20 is a view showing a first example of an imaging result acquired in a second step of a third imaging method;

FIG. 21 is a view showing a second example of an imaging result acquired in a second step of the third imaging method;

FIG. 22 is an expanded view showing a locking stent 2B according to a second embodiment expanded onto a plane virtually;

FIG. 23 is an enlarged view of a region B indicated in FIG. 22;

FIG. 24 is an expanded view showing a locking stent 2C according to a third embodiment expanded onto a plane virtually;

FIG. 25 is an expanded view showing a locking stent 2D according to a fourth embodiment expanded onto a plane virtually;

FIG. 26 is an expanded view showing a locking stent 2E according to a fifth embodiment expanded onto a plane virtually; and

FIG. 27 is an expanded view showing a locking stent 2F according to a sixth embodiment expanded onto a plane virtually.

### PREFERRED MODE FOR CARRYING OUT THE INVENTION

Hereinafter, embodiments of a distal stabilizer according to the present invention will be described. It should be noted that all of the drawings attached to the present specification are schematic diagrams, and the shape, the scale, the vertical and horizontal dimensional ratio, and the like of each part are changed or exaggerated from the actual ones in consideration of ease of understanding and the like. For example, a catheter or the like is shown to be shorter than actual in a longitudinal direction, and on the other hand, larger than actual in a radial direction. In this specification, etc., terms specifying a shape, a geometric condition, and degrees thereof, for example, terms such as “direction”, include a range generally regarded as the direction in addition to the strict meaning of the term. In addition, in the present specification, the long axis direction in a state where the distal stabilizer 1 extends linearly is also referred to as an “axial direction LD” or simply as an “axial direction”. In the axial direction LD, a proximal side close to a practitioner is referred to as “LD1”, and a distal side away from the practitioner is referred to as “LD2”. In addition, a direction orthogonal to the axial direction LD is described as a radial direction RD. Further, a direction orthogonal to the axial direction LD in an expanded view is described as a circumferential direction OD. The circumferential direction includes directions oblique to the radial direction RD, in addition to the radial direction RD.

### First Embodiment

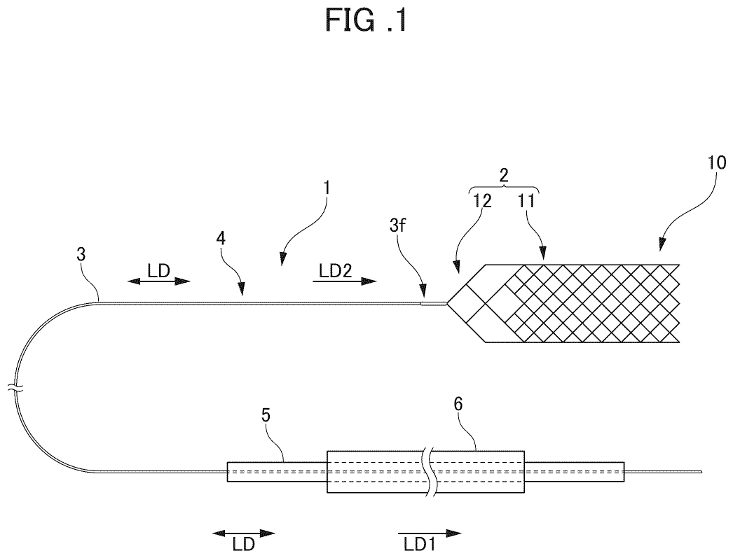

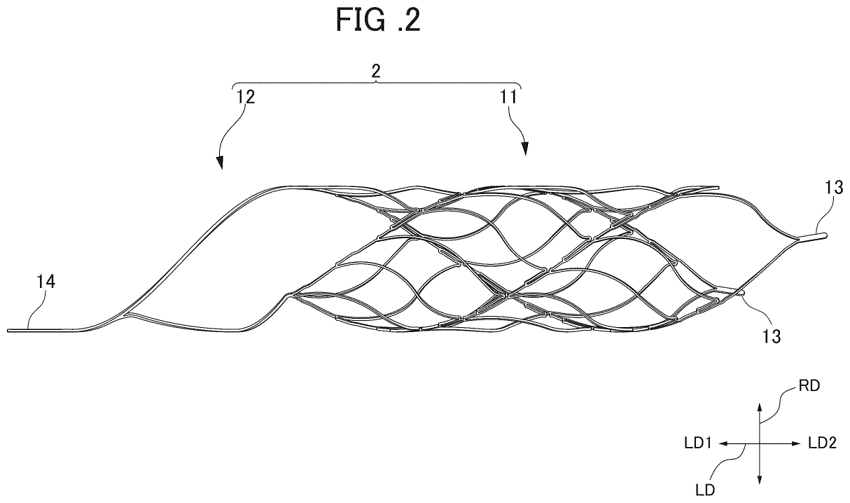

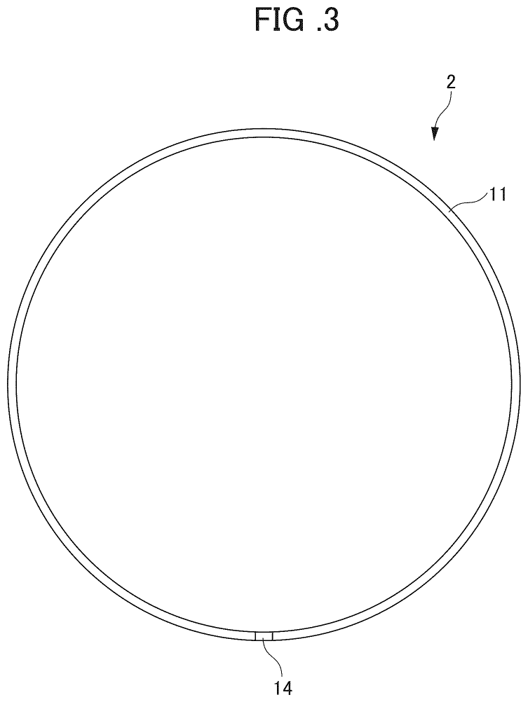

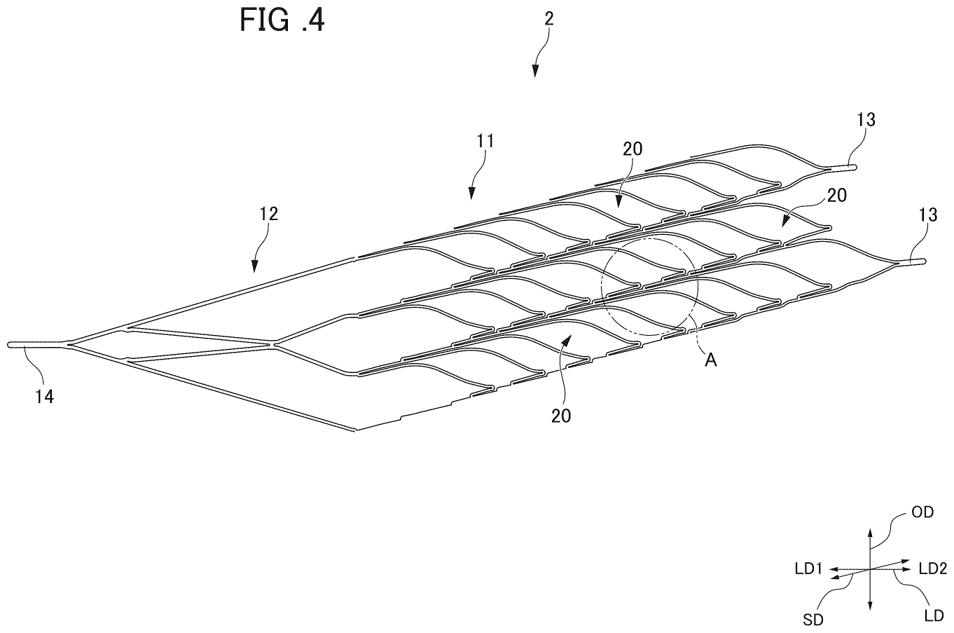

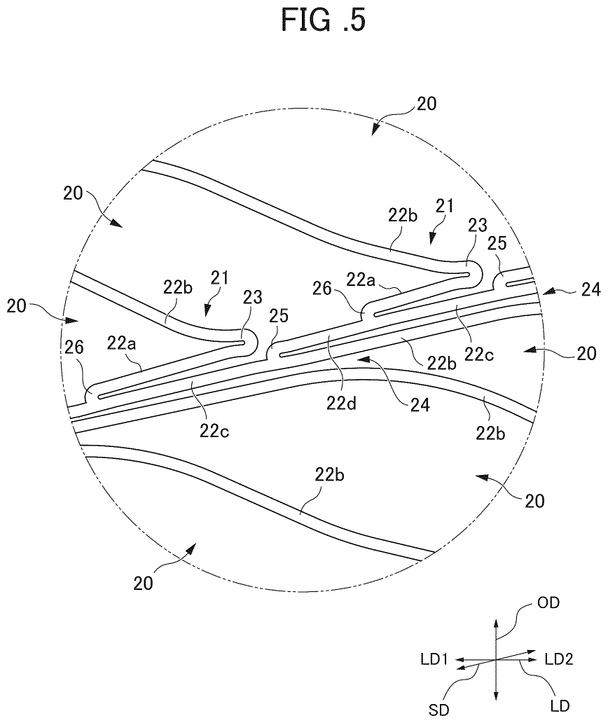

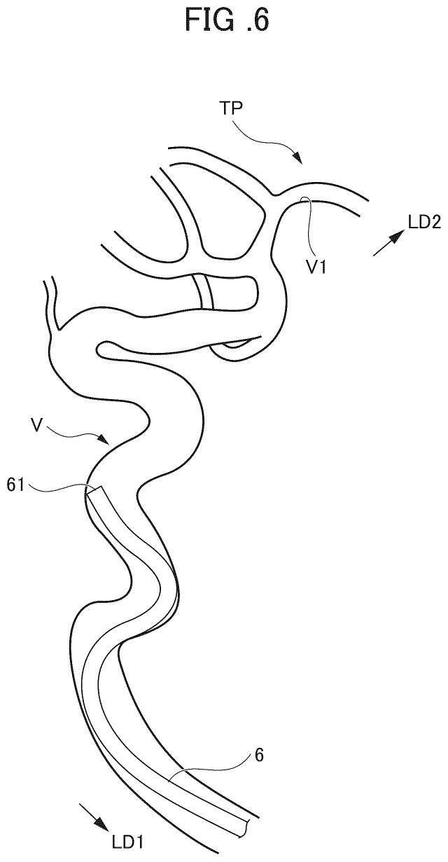

FIG. 1 is a view showing a delivery system 10 including a distal stabilizer 1 according to a first embodiment. FIG. 2 is a drawing viewing a diametrically expanded state of a locking stent 2 from a direction orthogonal to the axial direction LD. FIG. 3 is a drawing viewing the locking stent 2 from a proximal LD1 side. FIG. 4 is an expanded view showing the locking stent 2 expanded onto a plane virtually. FIG. 5 is an enlarged view of a region A indicated in FIG. 4. FIGS. 4 and 5 are expanded views in a natural state (described later) of the locking stent 2.

The delivery system 10 shown in FIG. 1, for example, can be used in the delivery of a treatment device in intracranial blood vessels of biological lumen such as an anterior cerebral artery (ACA), arteria communicans anterior (Acom) and middle cerebral artery (MCA). It should be noted that the blood vessels of biological lumen in which the delivery system 10 can be used are not limited to the above described examples. The blood vessels of biological lumen may be coronary arteries, blood vessels of upper and lower limbs (arteries and veins), organs and the like, in addition to the intracranial blood vessels. In the following description, biological lumen is also referred to as “blood vessel”.

As shown in FIG. 1, the delivery system 10 includes the distal stabilizer 1, and a plurality of catheters including a first catheter 5 and a second catheter 6. The first catheter 5, for example, is a catheter called a microcatheter. The second catheter 6 is placed over the first catheter 5. The second catheter 6 is a catheter of larger diameter than the first catheter 5. The diameter of the first catheter 5 is set according to a target position for delivering the second catheter 6, the inner diameter and the degree of bending of a biological lumen as a path to the target position. Although not limited, the inner diameter of the first catheter 5 may be preferably 0.017 inches or less, and more preferably 0.0165 inches or less. One or more other catheters (not shown) placed over the second catheter 6 may be used as necessary. In general, if more catheters are used, it may be possible to insert a catheter having a large inner diameter into a biological lumen and advance the catheter therein.

The distal stabilizer 1 is a device used for catheter delivery in a biological lumen. The distal stabilizer 1 includes the locking stent (cylindrical portion) 2 and a delivery wire (linear delivery member) 3.

The locking stent 2 is an anchor device which is inserted into the first catheter 5 in a diametrically reduced state, released from the first catheter 5 within a blood vessel to expand in diameter, and locked to the inner wall of a biological lumen. The locking stent 2 is coupled so as to extend from a distal end 3f of the delivery wire 3. As shown in FIG. 1, the locking stent 2 includes a cylindrical portion 11 and a converging portion 12. The cylindrical portion 11 is formed cylindrically as shown in FIG. 3, and includes a mesh pattern structure in which a plurality of cells 20 described later are arranged. In FIG. 1, the configuration of the cylindrical portion 11 is depicted by simplification. The converging portion 12 is a portion that converges a proximal D1 side of the cylindrical portion 11 toward the delivery wire 3.

As shown in FIG. 2, a distal marker 13 is provided at an end portion of the locking stent 2 on the distal LD2 side. A proximal marker 14 is provided at an end portion of the locking stent 2 on the proximal LD1 side. Each marker is made of a radiopaque material. The distal marker 13 provides a mark for confirming the position of the end portion of the locking stent 2 on the distal LD2 side. The proximal marker 14 provides a mark for confirming the position of the locking stent 2 on a proximal LD1 side.

The cylindrical portion 11 has a structure in which the plurality of cells 20 are arranged along the axial direction LD of the locking stent 2. In the present embodiment, the cylindrical portion 11 has a mesh pattern structure in which the plurality of cells 20 including open cell portions 21 and closed cell portions 24 described later are arranged in a spiral manner relative to the axial direction LD of the locking stent 2. As shown in FIG. 4, the plurality of cells 20 are arranged in a spiral manner along an arrangement direction SD of cells that is oblique to the axial direction LD (arrangement direction in the design of cells, and can also be called a spiral direction due to being arranged spirally relative to the axial direction LD). In the present embodiment, the arrangement direction SD of cells slopes obliquely downward-left from the distal LD2 side to the proximal LD1 side of the locking stent 2, as shown in FIG. 4; however, it is not limited thereto. The arrangement direction SD of cells may slope obliquely upward-left (reverse direction from FIG. 4) from the distal LD2 side to the proximal LD1 side of the locking stent 2. “Along arrangement direction SD of cells” refers to being parallel or substantially parallel to the arrangement direction SD of cells. “Substantially parallel”, for example, refers to a range of about 1 to 15° for the angle at which the arrangement direction of the cells 20 intersects with the arrangement direction SD of cells. In the locking stent 2, the angle at which the axial direction LD and the arrangement direction SD of cells intersect with each other may be desirably less than 45°.

Cell 20 is also called an opening or compartment, and refers to a portion surrounded by wire-shaped struts (wire-shaped members) 22 forming a mesh pattern of the cylindrical portion 11. An open cell portion 21 is a portion having a free convex end 23 of the cell 20. In the locking stent 2 of the present embodiment, every cell 20 is an open cell having an open cell portion 21. The free convex end 23 is an end portion at which the distal LD2 sides of two struts 22a, 22b connect with each other, and other struts do not connect, as shown in FIG. 5. The shape of the free convex end 23, for example, is a substantially V shape, substantially U shape, substantially Q shape, or the like. It may be acceptable if the projecting direction of the free convex end 23 is generally on the distal LD2 side and parallel with the arrangement direction SD of cells with respect to the direction in which the locking stent 2 is inserted into the catheter. The free convex end 23 is not constrained by other struts, and thus displacement or deformation in the radial direction (direction orthogonal to the axial direction LD) is hardly limited. It should be noted that the projecting direction of the free convex end 23 is not limited to the example of the present embodiment.

As shown in FIG. 5, among the struts 22a, 22b forming the free convex end 23, the strut 22a extending in the arrangement direction SD of cells is arranged to be substantially parallel to the strut 22c of another cell 20 adjacent side by side to the arrangement direction SD of cells. Extending in the arrangement direction SD of cells refers to extending substantially linearly in the arrangement direction SD of cells. A length L1 of the strut 22a extending in the arrangement direction SD of cells is 0.2 to 1.4 mm, for example. The length of a strut refers to a length of a portion in the extending direction of the strut excluding a portion linking with another strut. The portion linking with another strut, for example, is a free convex end 23, closed convex end 25, apex 26, etc. When a strut is non-linear or includes a non-linear portion, the length of a strut represents a virtual length in a state where the strut is extended virtually in a straight line. In addition, being adjacent side by side in the arrangement direction SD of cells refers to being adjacent in a direction orthogonal or substantially orthogonal to the arrangement direction SD of cells. Arranged “substantially parallel” refers to being arranged at an angle of 0° (parallel) or 15° or less.

As shown in FIG. 5, a strut 22c extends continuously on the proximal LD1 side of a strut 22b, and is provided in an area which is more proximal than a closed convex end 25 on the proximal LD1 side and more distal than an apex 26 on the distal LD2 side. A strut 22d extends continuously on the proximal LD1 side of a strut 22c, and is provided in an area which is more proximal than an apex 26 on the proximal LD1 side and more distal than a closed convex end 25 to the distal LD2 side. The proximal LD1 side of a strut 22a, the proximal LD1 side of a strut 22c and the distal LD2 side of a strut 22d are linked at an apex 26. The proximal LD1 side of a strut 22b, the proximal LD1 side of a strut 22d and the distal LD2 side of a strut 22d are linked at a closed convex end 25. In the arrangement direction SD of cells, the length L2 of a strut 22 d is 0.1 to 0.7 mm, for example. The length L1 of a strut 22a and the length L2 of a strut 22d may be the same for every cell, or may differ from each other for every cell. It should be noted that the ratio of the length of a strut 22a to the length of a strut 22d, for example, is preferably on the order of 2:1; however, it is not limited to this ratio. In addition, the ratio of the length of a strut 22a to the length of a strut 22c, for example, is preferably on the order of 1:1.2; however, it is not limited to this ratio.

Among the struts forming the free convex end 23, the strut 22a extending in the arrangement direction SD of cells, in the natural state, is arranged parallel (intersection angle of 0°) to: the strut 22c which is adjacent side by side relative to the arrangement direction SD of cells; and the strut 22b near the apex 26, or arranged so as to intersect at an angle of 15° or less. The natural state refers to a state in which the locking stent 2 does not reduce in diameter (unloaded state).

The strut 22a extending in the arrangement direction SD of cells is arranged substantially parallel with the strut 22c and the strut 22b near the apex 26. Accordingly, the surplus space between struts reduces and it is possible to arrange the struts closely side by side. For this reason, the strut 22b, in which surplus space remains, is folded back starting from a part lying away from the apex 26, when a diametrical reduction occurs. Accordingly, the diametrical reduction of the locking stent progresses while the struts 22a and 22c almost do not change the orientation relative to the arrangement direction SD of cells. The diametrical reduction is performed with the orientation of struts being mostly unchanged. Accordingly, it is possible to perform the diametrical reduction while keeping the struts substantially in parallel with each other. As a result, it is possible to perform the diametrical reduction without having the struts 22a and 22c intersect with each other in the radial direction. With such an effect described above, the locking stent 2 reduced in diameter and accommodated in the first catheter 5 excels in sliding relative to the inner wall of the first catheter 5, since the diameter hardly increases. Since the locking stent 2 excels in sliding property, pushing the locking stent 2 toward the distal LD2 side is facilitated. The locking stent 2 also excels in a resheathing property for the first catheter 5 after the second catheter 6 being delivered to the target position. In addition, in each cell 20 forming the cylindrical portion 11, the strut 22a aligns in parallel to the arrangement direction SD of cells. Since the driving force exerted through pushing by a practitioner tends to transmit in the arrangement direction SD of cells, the present configuration excels in deliverability of the locking stent 2 toward the distal LD2 side.

The closed cell portion 24 is a portion of the cell 20 having the closed convex end 25. The closed convex end 25 is an end portion at which the proximal LD1 sides of the struts 22b and 22d link with each other, as shown in FIG. 5. The closed convex end 25 is the end portion which does not form a free convex end since another strut 22c links at the end portion. It should be noted that the strut 22b forms a portion of the free convex end 23 on the distal LD2 side and a portion of the closed convex end 25 on the proximal LD1 side. The shape of the closed convex end 25, for example, is a substantially V shape, substantially U shape, substantially Q shape or the like. The projecting direction of the closed convex end 25 may direct roughly toward the proximal LD1 side. In this way, the closed convex end 25 of the closed cell portion 24 projects toward the proximal LD1 side. However, the outward flipping motion of the closed convex end 25 is restrained by the other strut 22c, so that it is unlikely to cause a hindrance when resheathing (accommodating) is performed into the first catheter 5.

The cylindrical portion 11 can be prepared by laser processing a tube formed from, for example, biocompatible materials, preferably from a superelastic alloy. In the case of preparing from a superelastic alloy tube, it is preferable to prepare by laser processing a tube of about 2 to 3 mm, followed by expanding to the desired diameter, and conducting a shape-memory treatment on the tube. The cylindrical portion 11 is not limited to laser processing and may also, for example, be prepared by: cutting processing or the like, ; and weaving into a cylindrical shape a metal wire molded into a wire shape.

Preferably, the material of the cylindrical portion 11 inherently has high rigidity and high biocompatibility. As such a material, for example, titanium, nickel, stainless steel, platinum, gold, silver, copper, iron, chromium, cobalt, aluminum, molybdenum, manganese, tantalum, tungsten, niobium, magnesium, calcium, alloys containing these, and the like may be exemplified. In addition, as such a material, for example, synthetic resin materials such as polyolefins such as polyethylene (PE) and polypropylene (PP), and polyamide, polyvinyl chloride, polyphenylene sulfide, polycarbonate, polyether, and polymethyl methacrylate may also be used. Furthermore, as such a material, for example, biodegradable resins (biodegradable polymers) such as polylactic acid (PLA), polyhydroxybutyrate (PHB), polyglycolic acid (PGA), poly-s-caprolactone, and the like may also be used.

Among the material described above, titanium, nickel, stainless steel, platinum, gold, silver, copper, magnesium or an alloy containing these may be desirable. As the alloy, for example, a Ni—Ti alloy, Cu—Mn alloy, Cu—Cd alloy, Co—Cr alloy, Cu—Al—Mn alloy, Au—Cd—Ag alloy, Ti—Al—V alloy or the like may be exemplified. In addition, as the alloy, alloys of magnesium with Zr, Y, Ti, Ta, Nd, Nb, Zn, Ca, Al, Li, Mn or the like may be exemplified. Among these alloys, a Ni-Ti alloy may be desirable.

In addition, the entirety of the locking stent 2 of the first embodiment including the cylindrical portion 11 and the converging portion 12 is configured as a radiographically visible part. Radiographically visible part serves as a mark for confirming the position or state of the locking stent 2 under X-ray observation within a biological lumen such as a blood vessel, and is formed from a material having high impermeability to X rays (high contrasting property). Herein, radiopacity indicates having high impermeability to X rays under observation using X rays during surgery, and being opaque to X rays to an extent that it is possible to observe the radiographically visible part as an image. “Impermeability to X rays” is necessary for obtaining “visibility” or “contrasting property” under observation using X rays.

Regarding the entirety of the locking stent 2 that serves as a radiographically visible part as in the present embodiment, there may be an example in which struts themselves are formed from a material having high radiopacity. In addition, as another configuration establishing the radiographically visible part, there may be an example in which the material having high radiopacity is formed in close contact with the entirety of the struts by plating, coating or the like.

The material having high radiopacity may be a metal, or may be a synthetic resin and, for example, gold, tantalum, platinum, tungsten, iridium, platinum-tungsten or the like, and alloy materials of these can be exemplified as the metal material. In addition, a polymer material having radiopacity with the addition of a radiopaque filler or the like may be exemplified.

In the present embodiment, a radiographic image is acquired by an X-ray imaging device (not shown). The X-ray imaging device is a device which acquires radiographic images as a moving image or a still image. A practitioner can visually recognize the behavior of the locking stent 2 within the biological lumen in real time, through a monitor screen on which a moving image of radiography acquired by the X-ray imaging device is projected. In addition, the practitioner can visually recognize the state of the locking stent 2 within the biological lumen through a monitor screen on which a still image of radiography acquired by the X-ray imaging device.

Returning to FIG. 1, the delivery wire 3 is a member that is used when the locking stent 2 is advanced or retracted within the biological lumen. The delivery wire 3 is delivered toward the distal LD2 side when the locking stent 2 is advanced within the biological lumen, and is drawn toward the proximal LD1 side when the locking stent 2 is retracted within the biological lumen. The delivery wire 3 is made of, for example, a metallic material having a high elastic modulus such as stainless steel. In addition, the diameter of the delivery wire 3 is not limited as long as it has physical properties sufficient to perform an advancing or retracting operation within the biological lumen and is adapted to the first catheter 5, and may be, for example, 0.005 to 0.018 inches. It should be noted that the linear delivery member is not limited to a metallic material such as a delivery wire, and may be made of, for example, a resin or a composite material of metal and resin.

Among the plurality of catheters including the second catheter 6, a catheter having an inner diameter larger than the outer diameter of the first catheter 5 is referred to as a treatment catheter. The treatment catheter is a catheter having an inner diameter sufficient to insert a treatment device therein or having an inner diameter sufficient to use itself as a treatment device. The treatment catheter may also be referred to as a guiding catheter in applications in which a treatment device is inserted therein. The treatment catheter itself may be used as a treatment device. In such an application, the treatment catheter may be called a thrombus aspiration catheter.

Next, a usage mode of a delivery system 10 including a distal stabilizer 1 of the present embodiment and an imaging method for the distal stabilizer 1 will be described. First, some procedures in an operation of delivering a treatment device to a target position TP using the delivery system 10 of the present embodiment will be described. Although various operations other than those described below are performed as the procedure, a description thereof is omitted here. In the present embodiment, a biological lumen V is a blood vessel. In particular, the distal stabilizer 1 and the delivery system 10 of the present embodiment are suitably used when a highly tortuous blood vessel is included in the blood vessel. Further, the distal stabilizer 1 and the delivery system 10 of the present embodiment are preferably used when the target position TP is located in a region of the blood vessel which has an inner diameter of 7 mm or less, specifically, less than 2.5 mm (preferably, 2.0 mm or less or 1.5 mm or less). Specifically, the target position TP may be located in a region after M2 (M2, M3, M4, etc.) of the middle cerebral artery (MCA), A1 and A2 regions of the anterior cerebral artery (ACA), a region after P1 (P1, P2, etc.) of the posterior cerebral artery (PCA), the internal carotid artery (ICA), etc. However, the target position TP is not limited thereto, and may be located in a wide range of inner diameters of 0.5 to 10 mm for a blood vessel.

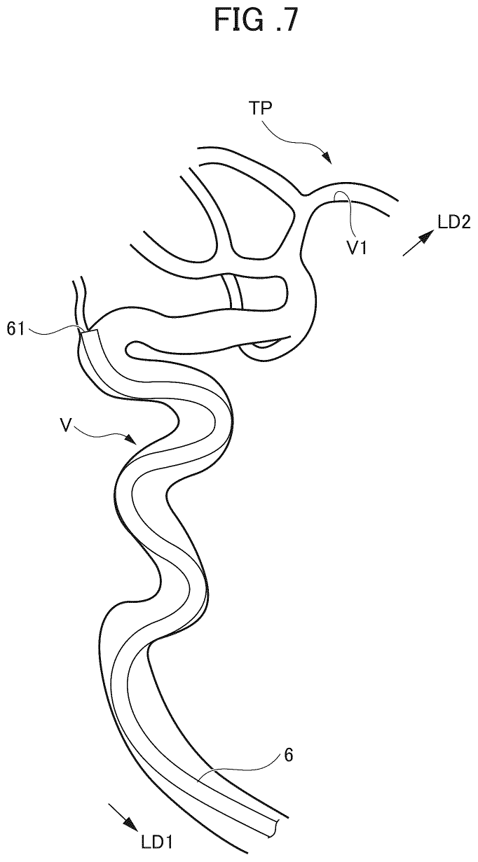

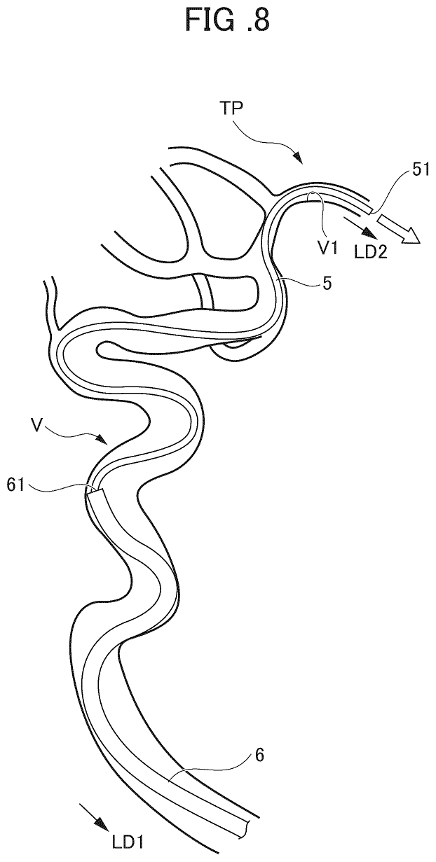

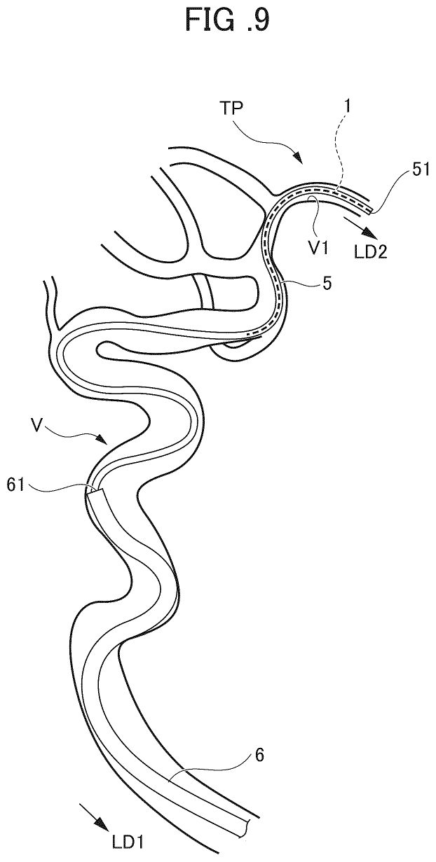

FIGS. 6 to 14 are schematic diagrams each illustrating an example of a procedure performed by the delivery system 10 including the distal stabilizer 1. First, a second catheter 6 is arranged on a proximal LD1 side of the biological lumen V of a patient. Typically, as shown in FIGS. 6 and 7, a distal end 61 of the second catheter 6 is caught by a bending portion or branching portion of the biological lumen V, and it is difficult to advance the second catheter 6 further on the distal side, accordingly. As shown in FIG. 8, the first catheter 5 is inserted into the second catheter 6 to be delivered into the biological lumen V, pushed out of the distal end 61 of the second catheter 6, and then a distal end 51 of the first catheter 5 is disposed in the vicinity of the target position TP. Subsequently, as shown in FIG. 9, the distal stabilizer 1 is inserted into the first catheter 5 and placed in the vicinity of the target position TP. At this time, a locking stent 2 of the distal stabilizer 1 is housed in the first catheter 5 in a diametrically reduced state. It should be noted that, in FIG. 9, the distal stabilizer 1 (the locking stent 2 and a delivery wire 3) is housed over the entire area of the first catheter 5; however, for convenience, the distal stabilizer 1 is illustrated by a broken line only on the distal side of the first catheter 5.

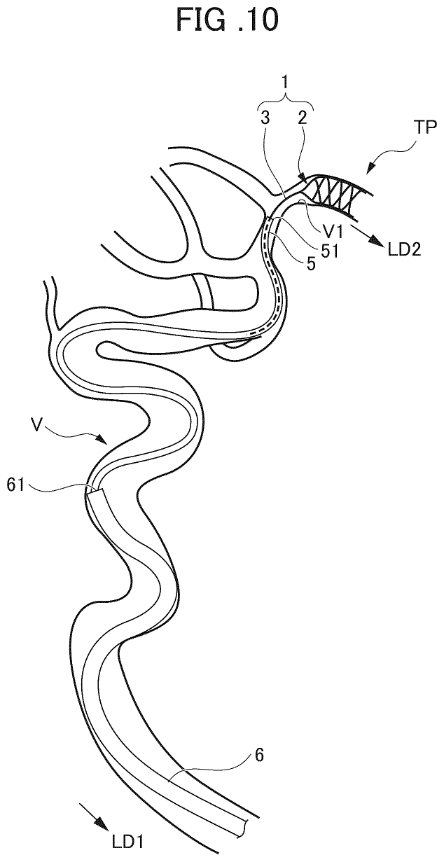

Next, as shown in FIG. 10, the locking stent 2 housed in the first catheter 5 in the diametrically reduced state is released from the distal end 51 of the first catheter 5. Release of the locking stent 2 is performed by an operation of retracting the first catheter 5 toward the proximal LD1 side. The locking stent 2 released from the distal end 51 of the first catheter 5 expands in diameter due to its self-expanding force. As a result, the locking stent 2 pushes an inner wall V1 of the biological lumen V from inside toward outside, and is locked to the inner wall V1.

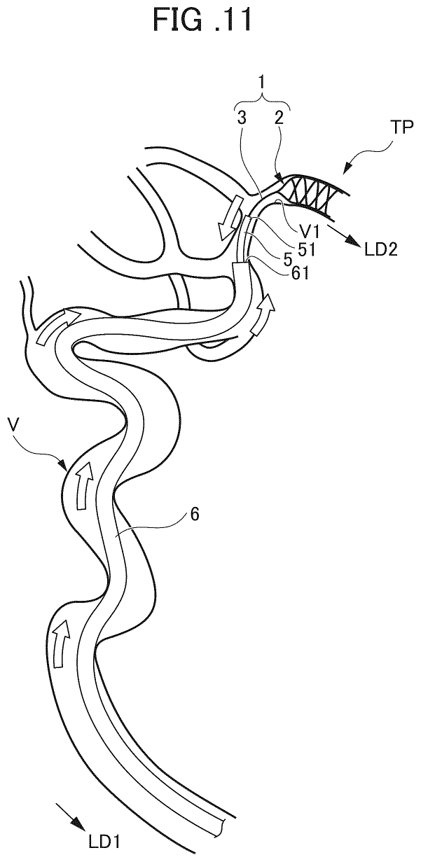

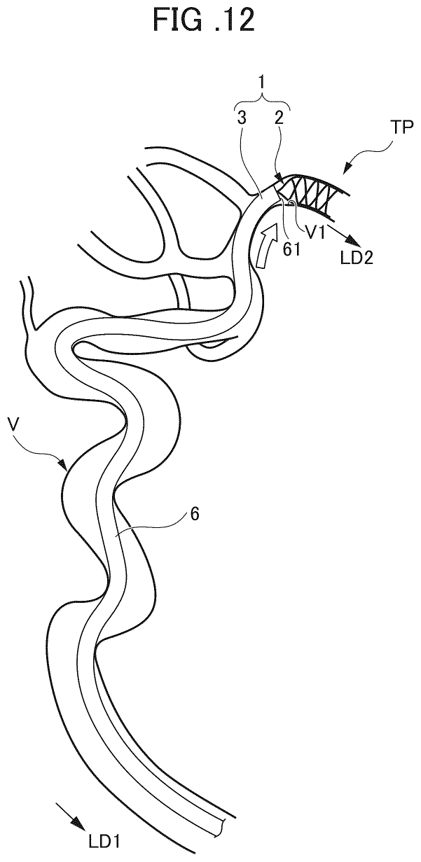

Subsequently, as shown in FIG. 11, the second catheter is placed over the first catheter 5, and the second catheter 6 is advanced toward the distal LD2 side within the biological lumen V. The second catheter 6 has a large outer diameter and high rigidity. For this reason, it may also be difficult to advance the second catheter 6. When it is difficult to advance the second catheter 6, an operation of pulling the delivery wire 3 toward the proximal LD1 side may be performed in a state where the locking stent 2 is locked to the inner wall V1 of the biological lumen V. By this pulling operation, the second catheter 6 can be advanced toward the distal LD2 side. Specifically, when the delivery wire 3 is pulled toward the proximal LD1 side in a state in which the locking stent 2 is locked to the inner wall V1, the path of the delivery wire 3 within the biological lumen V becomes close to a straight line and accordingly becomes short. Under the circumstance described above, while a portion of the proximal LD1 side of the second catheter 6 is held directly or indirectly, the distal end 61 of the second catheter 6 instead advances within the biological lumen V as shown in FIG. 12 since the position of the portion of the proximal LD1 side of the second catheter 6 does not change. Further, when the distal end 61 of the second catheter 6 advances into the biological lumen V without changing the position of the portion of the second catheter 6 on the proximal LD1 side, the path of the second catheter 6 becomes close to a straight line. This operation is particularly beneficial when the second catheter 6 attempts to pass through a difficult place to pass through, for example, a place where the bending of a biological lumen V is severe or a place where an inner diameter is small. As described later, according to the distal stabilizer 1 of the present embodiment, even in a case of performing such an operation, it is possible to grasp the degree of load acting on a cylindrical portion 11 since it is possible to confirm the shape of the cylindrical portion 11 by imaging a radiographically visible part.

In addition, when a treatment catheter having a large outer diameter is delivered to the vicinity of an aneurysm by locking the locking stent 2 to the distal LD2 side of the aneurysm, the first catheter 5 inserted into the treatment catheter (the second catheter 6) that is in the middle of advancement may stray into the aneurysm together with the delivery wire 3. In such a case, by performing an operation of pulling the delivery wire 3 toward the proximal LD1 side in a state where the locking stent 2 is locked to the distal side of the aneurysm, the delivery wire 3 and the first catheter 5 can be returned into the biological lumen V from the aneurysm since the path of the delivery wire 3 within the biological lumen V is shortened. As described above, even when the operation of bringing the wire or the catheter, which has strayed into the aneurysm, back into the biological lumen V is performed with the distal stabilizer 1 of the present embodiment, it is possible to confirm the shape of the cylindrical portion 11 by imaging the radiographically visible part, and thus to restrain the displacement of the locking stent 2 locked within the biological lumen V. Consequently, it is possible to suitably use in an application which delivers a treatment catheter to the vicinity of the target position in a state where the locking stent 2 is locked to the distal side of the aneurysm.

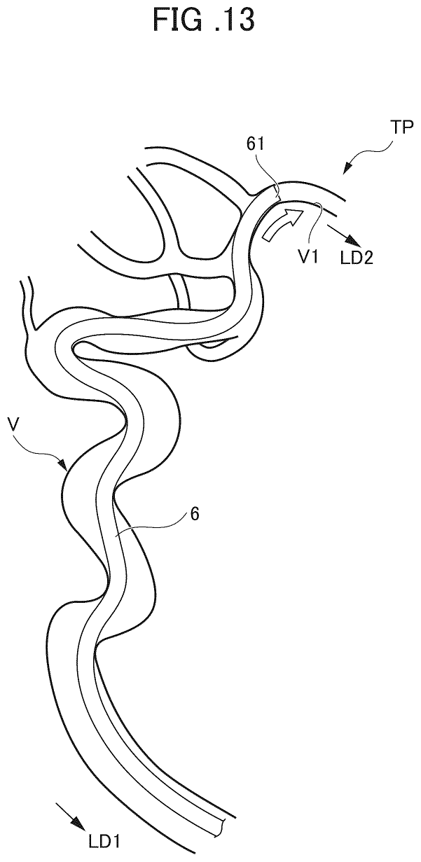

After the delivery of the second catheter 6 is completed in this manner, as shown in FIG. 13, the distal stabilizer 1 is retracted toward the proximal LD1 side and housed in the first catheter 5 (not shown). After the distal stabilizer 1 is housed in the first catheter 5, the first catheter 5 and the distal stabilizer 1 may be pulled out from the second catheter 6 on the proximal LD1 side. Alternatively, after the first catheter 5 is pulled out on the proximal LD1 side, the distal stabilizer 1 may be housed in the second catheter 6 and pulled out from the second catheter 6 on the proximal LD1 side. In the distal stabilizer 1 of the present embodiment, since the flexibility of a converging portion 12 on the proximal side is high, it is possible to facilitate housing (resheathing) into the first catheter 5 or the second catheter 6.

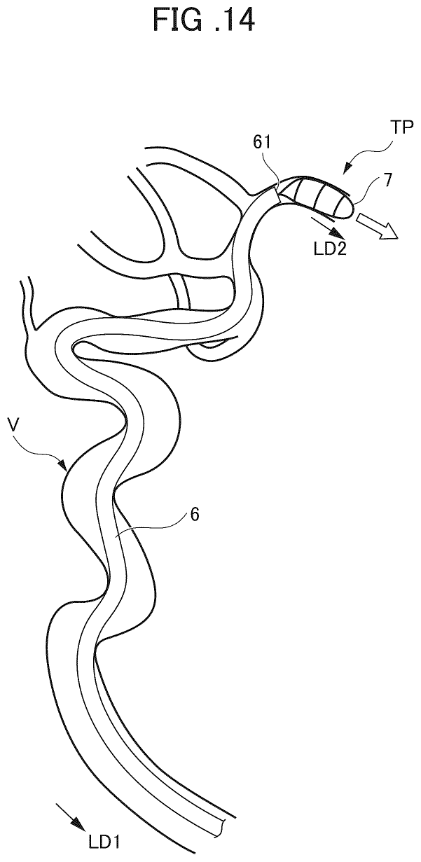

Next, as shown in FIG. 14, by providing the second catheter 6 at the target position TP within the biological lumen V, it is possible to easily deliver a treatment device 7, which is inserted into the second catheter 6, to the target position TP. Further, the second catheter 6 itself may be used as a treatment device. For example, when the second catheter 6 is used as a thrombus aspiration device, a thrombus at the target position TP is aspirated and removed from the distal end 61 by applying a negative pressure to the internal flow path of the second catheter 6. It should be noted that the timing for removal of the distal stabilizer 1 is not limited but may be either before or after the delivery of the treatment device 7. In the latter case, the second catheter 6 has at least a double or higher multiple structure of one lumen into which the distal stabilizer 1 and the first catheter 5 are inserted and another lumen into which the treatment device 7 is inserted.

The treatment device 7 is, for example, a thrombus aspiration device, a flow diverter, an aneurysm embolization device, an aneurysm treatment device, an intracranial arterial stenosis treatment device, a balloon catheter, a shunt, or a liquid embolic substance release means (such as a catheter with a lumen through which the liquid embolic substance passes). According to the distal stabilizer 1 of the present embodiment, these treatment devices can also be delivered, in terms of transcatheter, to the above-described region of inner diameters of 0.5 mm to 10 mm, preferably 7 mm or less, specifically less than 2.5 mm (preferably 2.0 mm or less, or 1.5 mm or less) for blood vessels. Diseases or conditions (vascular occlusions, aneurysms, etc.) in these regions can be treated or ameliorated with little burden on the patient.

Further, the treatment device 7 delivered to the target position TP may be used within the biological lumen (a thrombus aspiration device, a flow diverter, an aneurysm embolization device, a device for treating aneurysm, a device for treating intracranial arterial stenosis, a balloon catheter, or a means for releasing liquid embolic substance, etc.) or may be used by protruding from the biological lumen (such as a shunt). The distal stabilizer 1 of the present embodiment is also applied to a method to perform a therapeutic action of: delivering the treatment device 7 to the target position TP within the biological lumen V with the delivery system 10; and causing the treatment device 7 to protrude from the target position TP within the biological lumen V to outside the biological lumen V.

It is possible to perform such catheter delivery method and treatment method as described below with the aforementioned surgical method.

(Catheter delivery method 1) A delivery method of a catheter within a biological lumen using the distal stabilizer 1 of the present disclosure may be performed that includes steps as described below. In a first step: delivering the first catheter 5 by inserting the first catheter 5 into the second catheter 6 having a larger inner diameter than the first catheter 5; pushing the first catheter 5 out from a distal end of the second catheter 6, followed by arranging the distal end of the first catheter 5 in a vicinity of a target position. In a second step: housing the distal stabilizer 1 in a diametrically reduced state into the first catheter 5. In a third step: releasing the housed distal stabilizer 1 from the distal end of the first catheter 5 and locking the housed distal stabilizer 1 to an inner wall. In a fourth step: imaging a radiographically visible part (the entirety of locking stent 2 in the present embodiment, which includes radiographically visible parts 101, 102, 103, 104, 105, 106, 107, 108, 109, etc. in embodiments described later in a state placing the second catheter 6 over the linear delivery member 3 of the distal stabilizer 1 and a state locking the distal stabilizer 1 to the inner wall. In a fifth step: advancing the catheter including the second catheter 6 toward the distal side while continuing the fourth step, in a state locking the distal stabilizer 1 to the inner wall. And in a sixth step: reducing the diameter of the distal stabilizer 1 and resheathing the distal stabilizer 1 into either one of the catheters (first catheter 5, second catheter 6), and drawing out the distal stabilizer 1 outside from the biological lumen after the fifth step.

(Catheter delivery method 2) In addition, in (Catheter delivery method 1), the fifth step may be performed in a state placing the second catheter 6 over the first catheter 5.

(Catheter delivery method 3) In addition, in (Catheter delivery method 1), the fifth step may be performed in a state drawing out the first catheter 5.

(Treatment method) In addition, after the sixth step of the above-mentioned (Catheter delivery method 1) to (Catheter delivery method 3), it is possible to perform a treatment method with a therapeutic action by inserting the treatment device 7 into the second catheter 6, and bringing the treatment device 7 out from the distal end of the second catheter 6.

The locking stent 2 makes contact while pressing the inner wall of the biological lumen by self-expansion (diametrical expansion) within the biological lumen, whereby locking is performed. The pressing force with which the locking stent 2 presses the inner wall of the biological lumen decreases as the diametrical expansion progresses, and the pressing force reaches zero when the diametrical expansion is completed. Accordingly, in a situation in which the locking stent 2 completely expands in diameter, for example, the pressing force acting on the inner wall of the biological lumen decreases, or completely disappears, which makes it impossible for the locking stent 2 to perform sufficient locking. Consequently, the locking stent 2 is desirably used while contacting the inner wall of a biological lumen not in a state of complete diametrical expansion but rather in the middle of diametrical expansion. So long as in this state, locking will be appropriately performed.

For the locking stent 2 of the present embodiment, it is possible to grasp the state of diametrical expansion of the locking stent 2 through observation using X rays (imaging), since the entirety thereof serves as the radiographically visible part. Consequently, it is possible to determine whether or not the locking stent 2 is appropriately locked according to the state of diametrical expansion of the locking stent 2. For example, in case of the diameter of 4 mm when the cylindrical portion 11 of the locking stent 2 is completely expanded in diameter, it is considered that the cylindrical portion 11 is locked with a high possibility if the diameter thereof is less than 4 mm at a time of diametrical expansion within the biological lumen. In addition, if the diameter of the cylindrical portion 11 is 3 mm or less, the possibility of being locked is even higher, and it may be determined that the cylindrical portion 11 is reliably locked if the diameter thereof is 2 mm or less. Suppose the above example in which the diameter of the cylindrical portion 11 is 3 mm or less. When the expected expansive force is obtained, it may be determined that locking is appropriately performed so long as the outer diameter of the cylindrical portion 11 acquired through observation (imaging) using X-rays is 3 mm or less. Hereinafter, a description will be made calling the diametrically expanded state of the cylindrical portion 11 which reaches the limit of diametrical expansion at which such an expected expansive force is obtainable as “limit of diametrical expansion”. It should be noted that the diameter of the cylindrical portion 11 of the locking stent 2 within the biological lumen can be measured numerically with an observation device (imaging device) using X-rays. In addition, if the locking stent 2 which expands fully in diameter or is in the state of the limit of diametrical expansion is visually confirmed and memorized in advance, it is possible to determine easily whether the locking stent 2 is sufficiently locked according to the shape thereof within the biological lumen, which is acquired through imaging with X-rays.

As mentioned above, with the observation of an image acquired for the diametrically expanded state of the cylindrical portion 11 of the locking stent 2 in the biological lumen, it is possible to determine whether the locking stent 2 is appropriately locked. Accordingly, it is possible to conduct an appropriate medical treatment more safely. Hereinafter, specific imaging methods will be illustrated, and the usefulness from grasping the diametrically expanded state of the locking stent 2 will be described.

### (First Imaging Method)

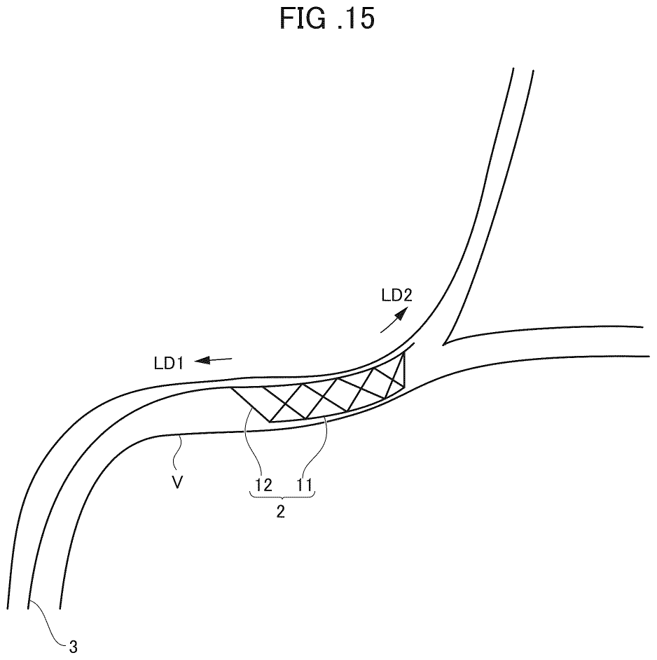

As a first imaging method, an imaging method and procedure will be described for a case where locking is insufficient, since the inner diameter of a blood vessel V at a position where a locking stent 2 diametrically expands is larger than expectation. FIG. 15 is a view showing imaging results acquired in a first step of the first imaging method. FIG. 15 shows the imaging results of the first step, for the case where the inner diameter of the blood vessel V at the position where the locking stent 2 diametrically expands is larger than the outer diameter of the cylindrical portion 11 of the locking stent 2 in the limit of diametrical expansion. In the case of using the locking stent 2, measurement of the blood vessel diameter at a target position is performed in advance. However, for example, there may also be cases where the measurement of the blood vessel diameter becomes inaccurate due to a small blood flow caused by vascular constriction. In such a case, a situation may arise where the inner diameter of the blood vessel is larger than expectation, which results in insufficient locking.

First, the first step of imaging the radiographically visible part is performed on a distal stabilizer 1 delivered together with a delivery wire 3 into a biological lumen. It should be noted that the entirety of the locking stent 2 in the present embodiment is the radiographically visible part. With the imaging results, it is possible to determine whether appropriate locking is being performed without the diametrical expansion of the locking stent 2 reaching the limit of diametrical expansion, or whether the diametrical expansion of the locking stent 2 exceeds the limit of diametrical expansion. When it is confirmed that appropriate locking is being performed without the diametrical expansion of the locking stent 2 exceeding the limit of diametrical expansion, it is possible to move on to a procedure such as delivering a treatment device to the target position using the locking by the locking stent 2.

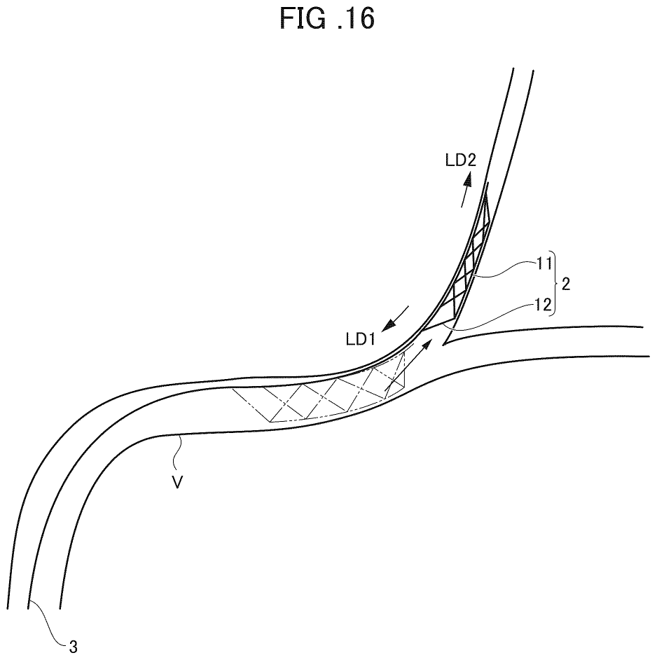

On the other hand, when it is determined with the imaging results acquired in the first step as shown in FIG. 15 that: the diametrical expansion of the locking stent 2 exceeds the limit of diametrical expansion; or the diametrical expansion of the locking stent 2 is close to the limit of diametrical expansion, it may be highly likely that locking is insufficient. In such cases, a second step of imaging the radiographically visible part is performed after moving the cylindrical portion 11 to a position more distal on the distal LD2 side of the biological lumen than the delivery position in the first step. Herein, in order to move the locking stent 2 to a new diametrically expanded position, the first catheter 5 is moved toward the distal LD2 side and the locking stent 2 is resheathed within the first catheter 5, and the first catheter 5 is further moved toward the distal LD2 side or the like. Then, by pushing the delivery wire 3 toward the distal LD2 side and moving the locking stent 2 to the new diametrically expanded position, followed by retracting the first catheter 5 toward the proximal LD1 side, the locking stent 2 is released from the first catheter 5 and allowed to expand in diameter as in FIG. 16.

FIG. 16 is a view showing an imaging result acquired in the second step of the first imaging method. The imaging results of the second step in FIG. 16 are acquired by imaging the radiographically visible part after moving the cylindrical portion 11 to a position within the biological lumen at which the diametrical expansion of the cylindrical portion 11 is more restrained than the diametrical expansion thereof which is visually recognized with the imaging results of the first step. So long as it is confirmed that the cylindrical portion 11 is sufficiently in a diametrically reduced state as in FIG. 16, it is possible to determine that the locking stent 2 performs locking appropriately, and move on to a procedure such as delivering a treatment device to a target position. Even if the inner diameter of the blood vessel V at the position where the locking stent 2 expands in diameter is larger than expectation and locking is insufficient, it is possible for the first imaging method to perform locking again at an appropriate position.

### (Second Imaging Method)

As a second imaging method, an imaging method will be described that establishes a criterion for the tensile load for the case of performing a procedure of drawing a delivery wire 3 toward a proximal LD1 side while a locking stent 2 is locked to the inner wall of a blood vessel V. In the state locking the locking stent 2 to the inner wall of the blood vessel V, a procedure of drawing the delivery wire 3 toward the proximal LD1 side may be performed. For example, in the middle of delivering a second catheter 6 toward the distal LD2 side along the delivery wire 3, the second catheter 6 may be caught at a branching point of blood vessels, a location where the curvature of a blood vessel is severe, or the like. In such a case, when delivering the second catheter 6 toward the LD2 side while drawing the delivery wire 3 toward the proximal LD1 side, it may be possible to deliver without the second catheter 6 being caught. However, if the tensile load drawing the delivery wire 3 toward the proximal LD1 side is too large, there is a risk that the delivery wire 3 is unlocked and the delivery wire 3 causes damage to the inner wall of the blood vessel V. Accordingly, it is important to establish an appropriate tensile load drawing the delivery wire 3 toward the proximal LD1 side.

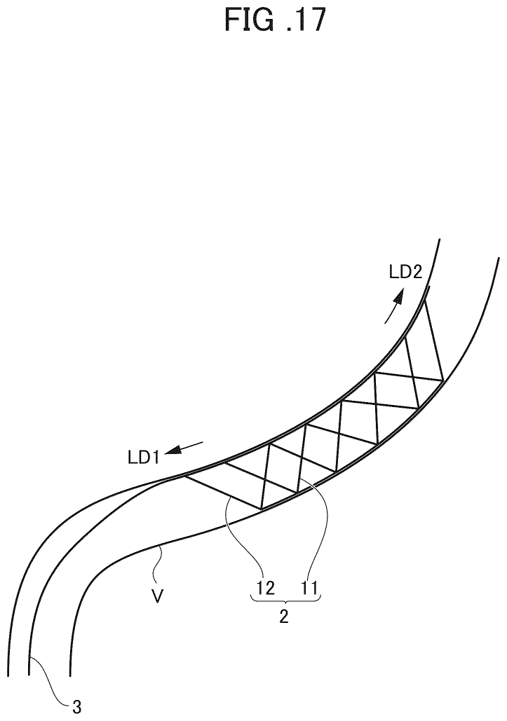

The second imaging method described below is useful to set the appropriate tensile load drawing the delivery wire 3 to the proximal LD1 side. In the second imaging method, as a first step, a radiographically visible part (entire locking stent 2 in present embodiment) is imaged before the operation of drawing the delivery wire 3 for the distal stabilizer 1 delivered together with the delivery wire 3 within the blood vessel. FIG. 17 is a view showing the imaging results acquired in the first step of the second imaging method. Herein, it is assumed that it can be confirmed with the imaging results of this FIG. 17 that a cylindrical portion 11 does not expand in diameter to exceed the limit of diametrical expansion, and is locked appropriately to the inner wall of the blood vessel V.

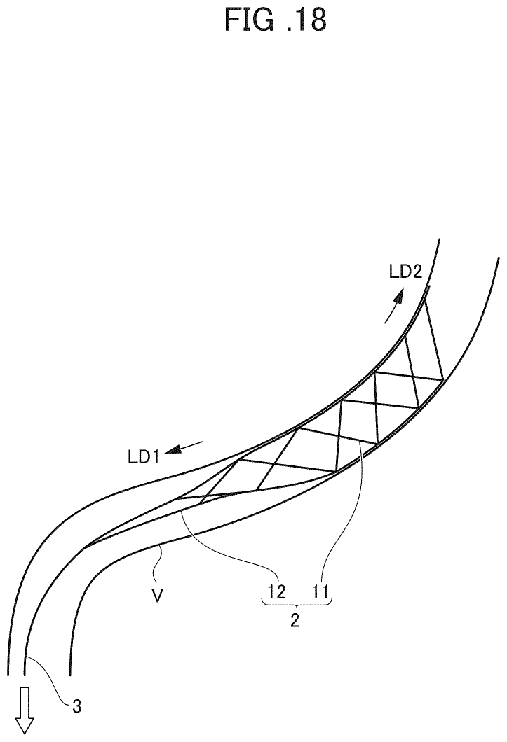

Next, after or during performing the operation of drawing the delivery wire 3 toward the proximal LD1 side, the second step of imaging the radiographically visible part is performed. FIG. 18 is a view showing the imaging results acquired in the second step of the second imaging method. As shown in FIG. 18, by the operation of drawing the delivery wire 3 toward the proximal LD1 side, the locking stent 2 extends in an LD direction and deforms with the outer diameter tapering on the proximal LD1 side (converging portion 12 side).

Since the shape of the cylindrical portion 11 can be confirmed with the imaging results acquired in the second step, it is possible to grasp an extent of load acting on the cylindrical portion 11. For example, when only the vicinity of the converging portion 12 is narrowed, it is possible to confirm that locking to the inner wall of the blood vessel V is maintained by the remaining cylindrical portion 11. Consequently, it is possible to determine that the load acting on the delivery wire 3 is suitable. In addition, when the outer diameter of the cylindrical portion 11 shrinks with respect to the range of a predetermined value (for example, 50% of the effective length) or less on the proximal LD1 side of the cylindrical portion 11, it is possible to determine that the load acting on the delivery wire 3 is suitable. On the other hand, when the outer diameter shrinks with respect to the range of a predetermined value (for example, 50% of the effective length) or more on the proximal LD1 side of the cylindrical portion 11, for example, it is possible to determine that the load acting on the delivery wire 3 is excessive. Typically, if the entirety of the cylindrical portion 11 narrows in diameter, it is possible to determine that the load acting on the delivery wire 3 is excessive. Herein, the effective length indicates a length along the axial direction LD of a cylindrical portion of the cylindrical portion 11, and the converging portion 12 is not included. When it is determined that the load acting on the delivery wire 3 is excessive, for example, it is better to reduce the load acting on the delivery wire 3 so that the range in which the outer diameter shrinks meets the predetermined value (for example, 50% of the effective length) or less on the proximal LD1 side of the cylindrical portion 11.

In this way, according to the second imaging method, it is possible to simultaneously use the imaging results acquired in the second step as a criterion to see how much tensile load drawing the delivery wire 3 toward the proximal LD1 side is applied when the locking stent 2 which is locked to the inner wall of the blood vessel V is in use.

### (Third Imaging Method)

As a third imaging method, an imaging method will be described that confirms the diametrically expanded state of a cylindrical portion 11 when a locking stent 2 which is locked to the inner wall of a blood vessel V shifts from an initially locked position. The position at which the locking stent is locked to the inner wall of the blood vessel V is desirably maintained at the initially locked position essentially without movement. However, for example, it is expected that the position of the locking stent 2 shifts in the aforementioned operation of drawing a delivery wire 3 toward a proximal LD1 side, or the position of the locking stent 2 shifts due to the initial locking being insufficient or the like. In such a case, it is important to grasp the state of the cylindrical portion 11 at a location to which the locking stent 2 has moved due to shifting.

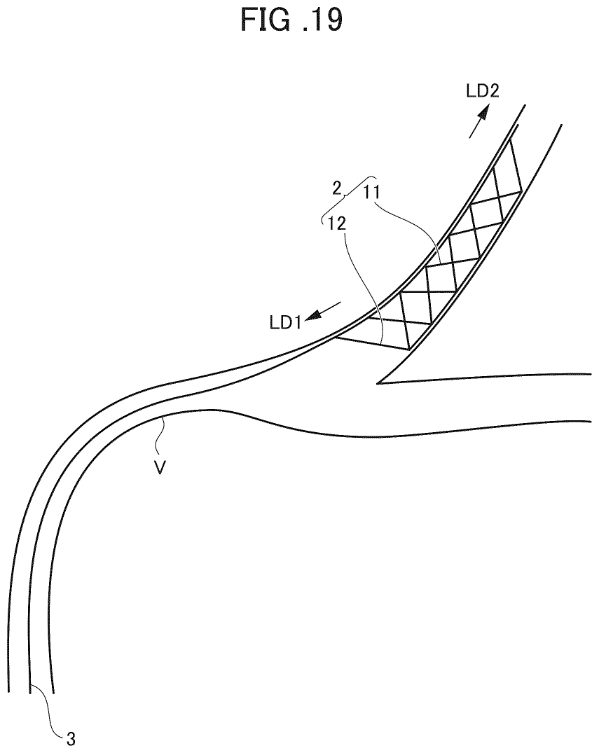

The third imaging method described below is useful in order to grasp the state of the cylindrical portion 11 at the point to which the locking stent 2 moved due to shifting. In the third imaging method, as a first step, the radiographically visible part (entire locking stent 2 in the present embodiment) is imaged for a distal stabilizer 1 delivered together with the delivery wire 3 into the blood vessel. FIG. 19 is a view showing an imaging result acquired in the first step of the third imaging method. Herein, it is assumed that it is possible to confirm with imaging results of FIG. 19 that the cylindrical portion 11 is locked to the inner wall of the blood vessel V.

With the imaging results acquired in the first step, it has been confirmed that the cylindrical portion 11 is locked to the inner wall of the blood vessel V. However, as described above, the cylindrical portion 11 within the blood vessel V may shift for some reason from a position thereof at the time of imaging in the first step. In such a case, the second step of imaging the radiographically visible part is performed after the cylindrical portion 11 within the blood vessel V has shifted from the position thereof at the time of imaging in the first step.

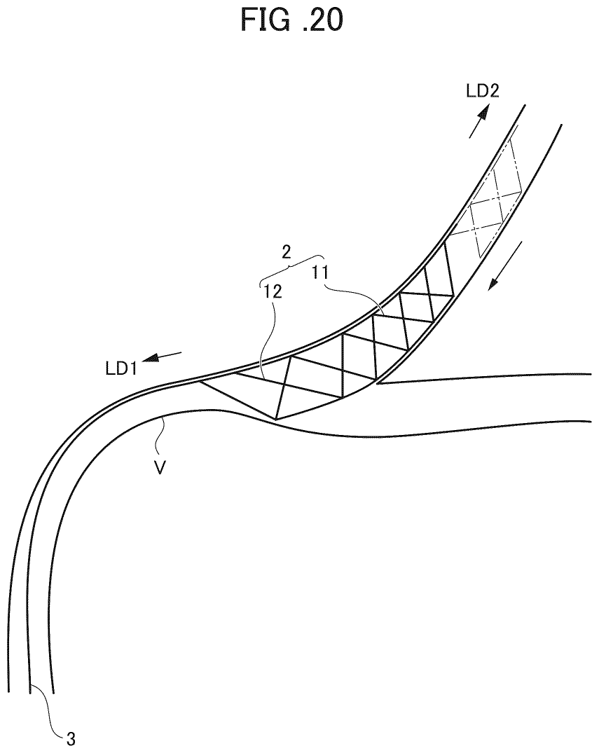

FIG. 20 is a view showing a first example of the imaging results acquired in the second step of the third imaging method. The first example shown in FIG. 20 shows an example in which the positional displacement of the locking stent 2 is not so large. Even in this case of the displacement being small as in FIG. 20, there may be cases where the relationship between the inner diameter of the blood vessel V and the outer diameter of the cylindrical portion 11 is no longer suitable for locking, depending on the shape of the blood vessel V. For example, when a part of the cylindrical portion 11 does not contact appropriately with the inner wall of the blood vessel V as in FIG. 20, it is possible to determine that there is a risk of the locking stent 2 not being appropriately locked. It should be noted that, it is determined that there is a risk of the locking stent 2 not being appropriately locked, it is desirable to start over the procedure of resheathing and locking the locking stent 2 within a first catheter 5.

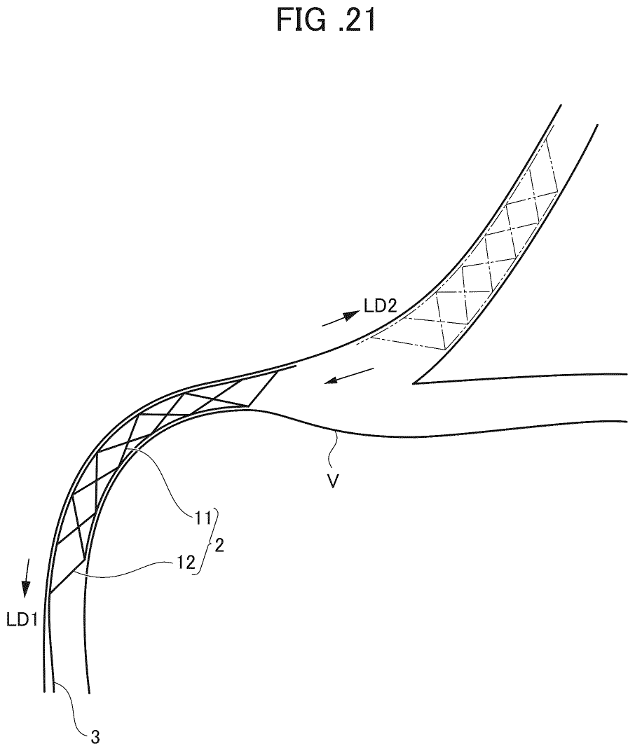

FIG. 21 is a view showing a second example of imaging results acquired in the second step of the third imaging method. The second example shown in FIG. 21 shows an example in which the positional displacement of the locking stent 2 is relatively large. Even if the position of the locking stent 2 greatly shifts as in FIG. 21, it is possible to determine that there is a sufficient locking force when the cylindrical portion 11 does not expand in diameter to exceed the limit of diametrical expansion. Consequently, so long as it is possible to confirm that locking is performed appropriately to the inner wall of the blood vessel V through confirming the diametrically expanded state of the cylindrical portion 11 with the imaging results acquired in the second step as in FIG. 21, it is possible to continue the subsequent procedure. As described above, according to the third imaging method, it is possible to confirm that the diametrically expanded state of the cylindrical portion 11 for the case where the locking stent 2 has shifted from an initially locked position, and accordingly it is possible to determine the next action appropriately. Herein, the next action is an action of starting over a procedure of resheathing and locking the locking stent 2 within the first catheter 5, and an action of performing a procedure of delivering a treatment device to a target position, as described above.

According to the present embodiment as described above, since the entirety of the locking stent 2 including the cylindrical portion 11 is configured as the radiographically visible part, it is possible to easily grasp the diametrically expanded state of the locking stent 2. In addition, if the first to third imaging method are carried out on an as-needed basis, it is possible to confirm the diametrically expanded state of the locking stent 2 at an appropriate time, and accordingly it is possible to allow the locking stent 2 to lock securely in many situations.

### Second Embodiment

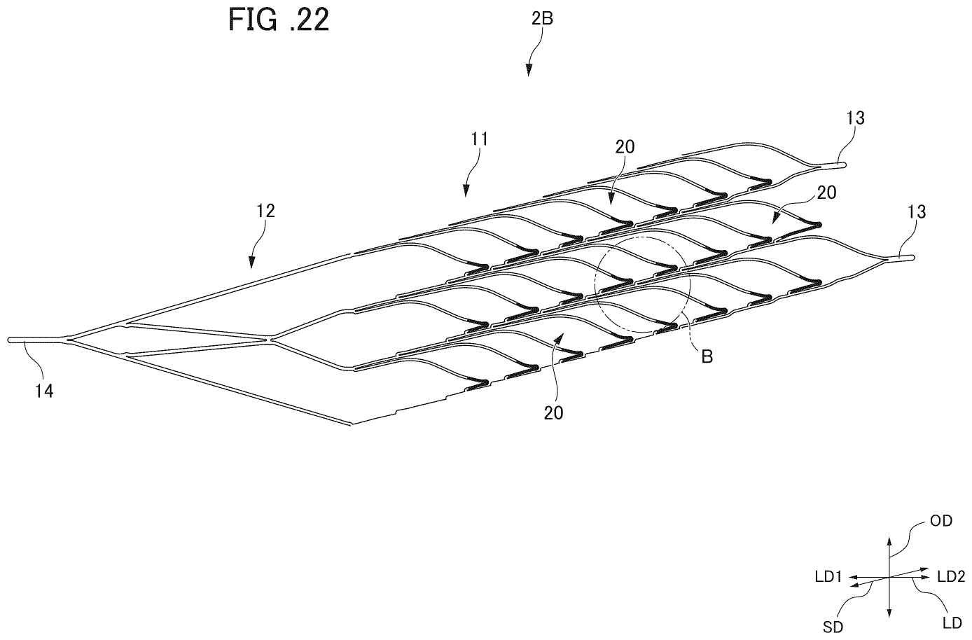

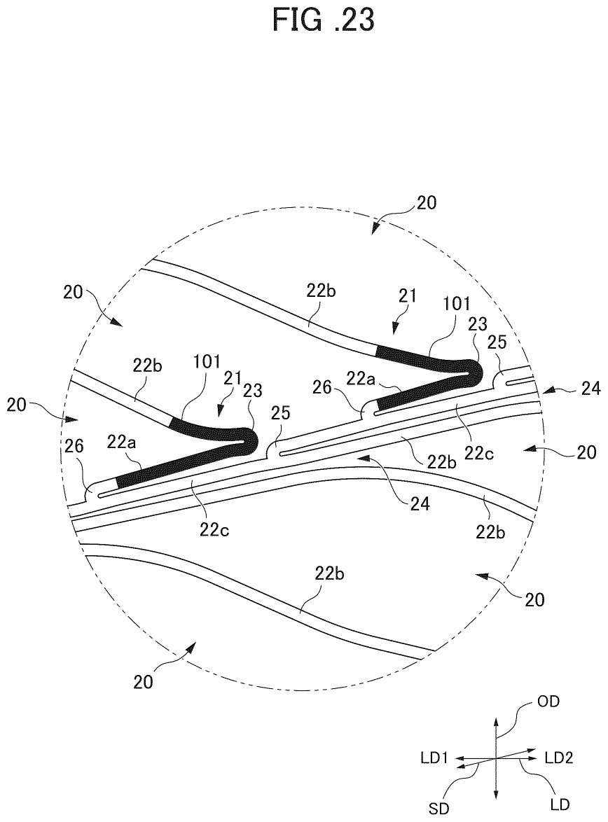

FIG. 22 is an expanded view showing a locking stent 2B of the second embodiment expanded onto a plane virtually. FIG. 23 is an enlarged view of a region B indicated in FIG. 22. A distal stabilizer 1 of the second embodiment differs in which portion of a locking stent 2B a radiographically visible part is provided from a distal stabilizer 1 of the first embodiment in which the entirety of a locking stent 2 is configured as a radiographically visible part. Other aspects of the distal stabilizer 1 according to the second embodiment are similar to the distal stabilizer 1 of the first embodiment. Consequently, the same reference numbers are assigned to portions achieving similar functions as the aforementioned first embodiment, and otherwise redundant descriptions will be omitted as appropriate.

In the first embodiment, the entirety of the locking stent 2 is configured as the radiographically visible part. However, when the entirety of the locking stent 2 is configured as the radiographically visible part, the physical characteristics of the locking stent 2 are affected by materials having high radiopacity. For example, when a material having high radiopacity is attached to the entirety of the locking stent 2 by plating, coating or the like, the locking stent 2 tends to be harder than before attachment, and there may be a risk of reduction in the followability to the shape of a blood vessel. So long as the radiographically visible part is provided with the object of making the diametrical expansion of cylindrical portion 11 visible, it may be sufficient to provide a radiographically visible part at a location where the diametrical expansion of the cylindrical portion 11 visually recognizable without configuring the entirety of the locking stent 2 as the radiographically visible part. More specifically, it may be better to provide the radiographically visible part to at least part of an area in which cells 20 of the cylindrical portion 11 are provided. Herein, the area in which the cells 20 are provided represents an area in which struts (wire-shaped members) are arranged around the cell 20. When only a portion of the struts arranged around the cell 20 is provided with the radiographically visible part, it is possible to grasp the diametrical expansion of the cylindrical portion 11 since the radiographically visible part displaces in response to deformation of the cell 20.

Accordingly, in the second embodiment, by providing the radiographically visible part to a part of the locking stent 2B (location enabling visible recognition of the diametrical expansion of the cylindrical portion 11), visibility is ensured while maintaining the followability of the locking stent 2B to the shape of a blood vessel.

In the second embodiment, as shown in FIGS. 22 and 23, a radiographically visible part 101 is provided to two struts 22a, 22b forming a free convex end 23 of an open cell portion 21. Particularly the vicinity of the free convex end 23 of the open cell portion 21 is a location that is not constrained by other struts and has high followability to the shape of a blood vessel. Consequently, even if the radiographically visible part 101 is provided to this portion by plating or coating the material having high radiopacity, there may be little affect adversely on followability to the shape of a blood vessel. In addition, since free convex ends 23 are arranged over the entirety of the locking stent 2B, radiographically visible parts 101 are provided over the entirety of the locking stent 2B, accordingly. Consequently, the diametrically expanded state of the locking stent 2B can be easily grasped similarly to the first embodiment, and the first imaging method to third imaging method can also be conducted.

### Third Embodiment

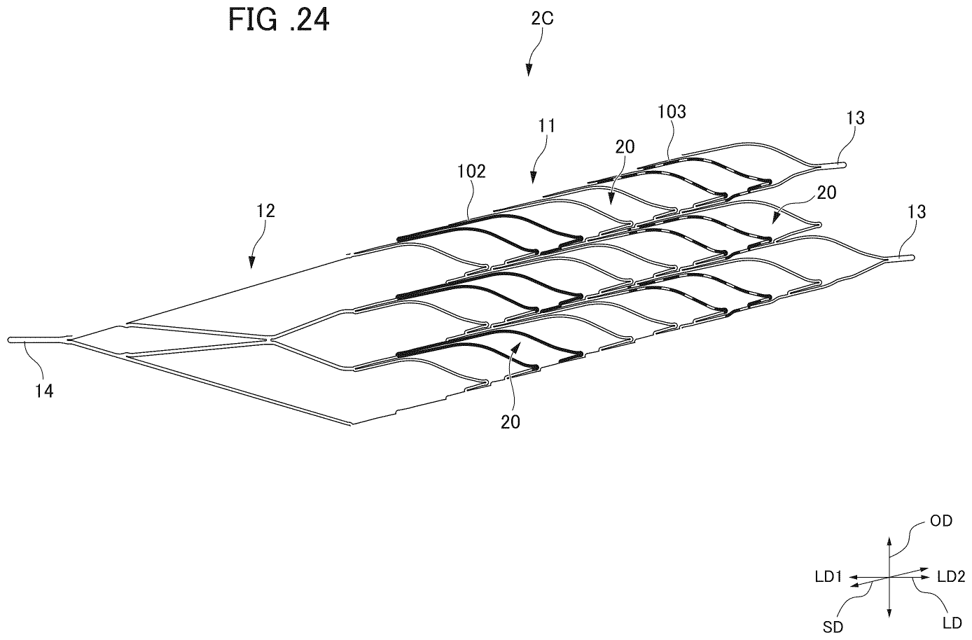

FIG. 24 is an expanded view showing a locking stent 2C according to a third embodiment expanded onto a plane virtually. A distal stabilizer 1 according to the third embodiment is similar to a distal stabilizer 1 of the second embodiment, except for differences in ashape and location where a radiographically visible part 102 or 103 is provided. Consequently, the same reference symbols are attached to portions functioning similarly to the aforementioned second embodiment, and otherwise redundant descriptions will be omitted as appropriate.

The locking stent 2C according to the third embodiment is provided with the radiographically visible part 102 (or radiographically visible part 103) at a strut surrounding a cell 20 aligned in a circumferential direction OD. FIG. 24 shows both the radiographically visible part 102 provided continuously along a strut, and the radiographically visible part 103 provided discontinuously like a broken-line along a strut. FIG. 24 illustrates the radiographically visible parts 102 and 103 mixed for explanation. And a radiographically visible part 102 may replace a radiographically visible part 103, and vice versa. In addition, the radiographically visible parts 102 (or radiographically visible parts 103) may be provided in a plurality of rows (rows aligned in the circumferential direction OD) as shown in FIG. 24, or may be in one row.

Since the locking stent 2C of the third embodiment is provided with the radiographically visible parts 102 (or radiographically visible parts 103) in rows aligned in the circumferential direction OD, the diametrically expanded state of the locking stent 2C can be visibly recognized in X-ray observation. In addition, by providing the radiographically visible parts 102 (or radiographically visible parts 103) in a plurality of rows (rows aligned in the circumferential direction OD), it is possible to grasp the diametrically expanded state for every position in an axial direction LD.

### Fourth Embodiment

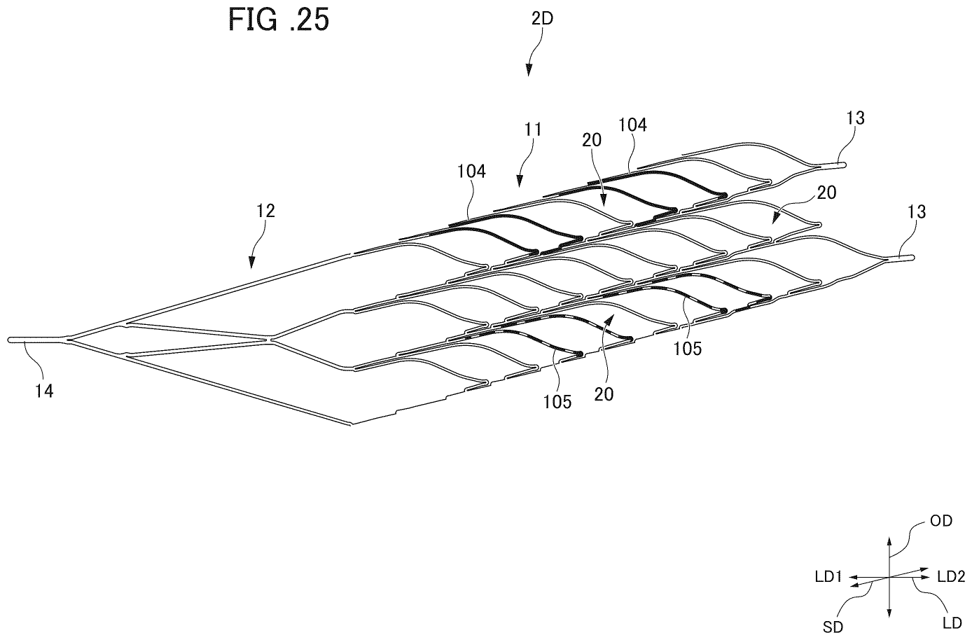

FIG. 25 is an expanded view showing a locking stent 2D according to the fourth embodiment expanded onto a plane virtually. A distal stabilizer 1 according to the fourth embodiment is similar to a distal stabilizer 1 of the second embodiment, except for differences in a shape and location where a radiographically visible part 104 or 105 is provided. Consequently, the same reference symbols are attached to portions functioning similarly to the aforementioned second embodiment, and otherwise redundant descriptions will be omitted as appropriate.

In the locking stent 2D of the fourth embodiment, the radiographically visible parts 104 (or radiographically visible parts 105) are provided to struts surrounding a plurality of cells 20 aligned in an arrangement direction SD. FIG. 25 shows both a radiographically visible part 104 provided continuously along a strut, and radiographically visible parts 105 provided discontinuously like a broken-line along a strut. FIG. 25 illustrates the radiographically visible parts 104 and 105 mixed for explanation. And a radiographically visible part 104 may replace a radiographically visible part 105, and vice versa. In addition, the radiographically visible parts 104 (or the radiographically visible parts 105) may be provided in a plurality of rows (rows aligned in the arrangement direction SD) as shown in FIG. 25, or may be in one row. In addition, the radiographically visible parts 104 (or the radiographically visible parts 105) may be spaced in the arrangement direction SD as shown in FIG. 25, or may be aligned continuously.

Since the locking stent 2D of the fourth embodiment is provided with the radiographically visible parts 104 (or the radiographically visible parts 105) in rows aligned in the arrangement direction SD, the diametrically expanded state of the locking stent 2D can be visibly recognized in X-ray observation. In addition, since the radiographically visible parts 104 (or the radiographically visible parts 105) are also arranged spirally, when viewed in the axial direction LD, it is possible to grasp the diametrically expanded state for every position in the axial direction LD.

### Fifth Embodiment

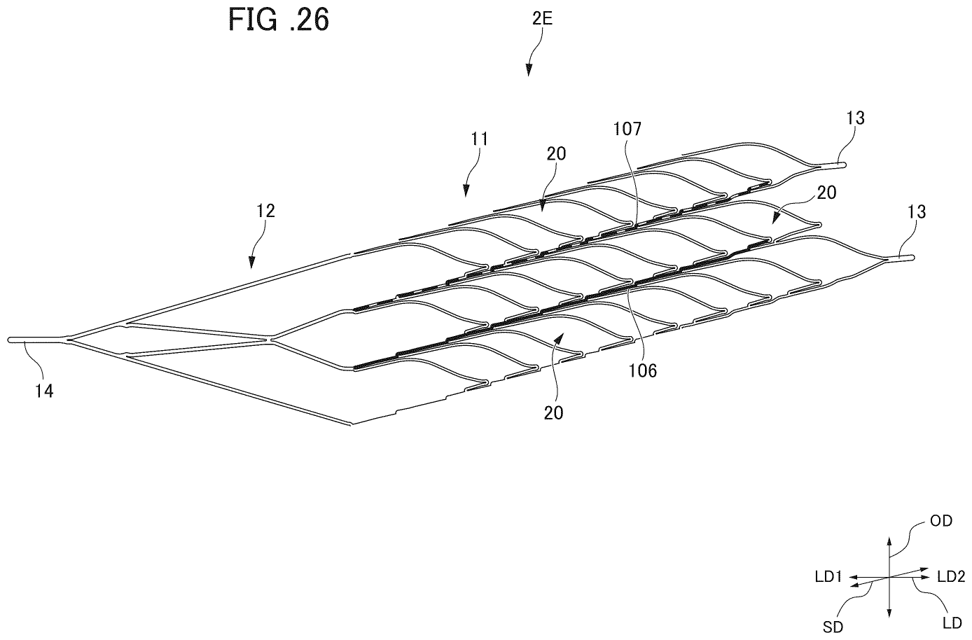

FIG. 26 is an expanded view showing a locking stent 2E according to a fifth embodiment expanded onto a plane virtually. A distal stabilizer 1 according to the fifth embodiment is similar to a distal stabilizer 1 of the second embodiment, except for differences in a shape and location where a radiographically visible part 106 or 107 is provided. Consequently, the same reference symbols are attached to portions functioning similarly to the aforementioned second embodiment, and otherwise redundant descriptions will be omitted as appropriate.

In the locking stent 2E according to the fifth embodiment, the radiographically visible part 106 (or the radiographically visible part 107) is provided to a strut extending substantially linearly in an arrangement direction SD in the expanded state shown in FIG. 26. FIG. 26 shows both a radiographically visible part 106 provided continuously along a strut, and radiographically visible parts 107 provided discontinuously like a broken-line along a strut. FIG. 26 illustrates the radiographically visible parts 106 and 107 mixed for explanation. And a radiographically visible part 106 may replace a radiographically visible part 107, and vice versa. In addition, the radiographically visible part 106 (or the radiographically visible part 107) may be provided in a plurality of rows as shown in FIG. 26, or may be in one row.

Since the locking stent 2D of the fifth embodiment in which the radiographically visible part 106 (or radiographically visible part 107) is provided to the strut extending substantially linearly in the arrangement direction SD makes the diametrically expanded state of the locking stent 2D visibly recognizable in X-ray observation. In addition, since the radiographically visible part 106 (or radiographically visible part 107) is also arranged spirally, when viewed in the axial direction LD, it is possible to grasp the diametrically expanded state for every position in an axial direction LD.

### Sixth Embodiment

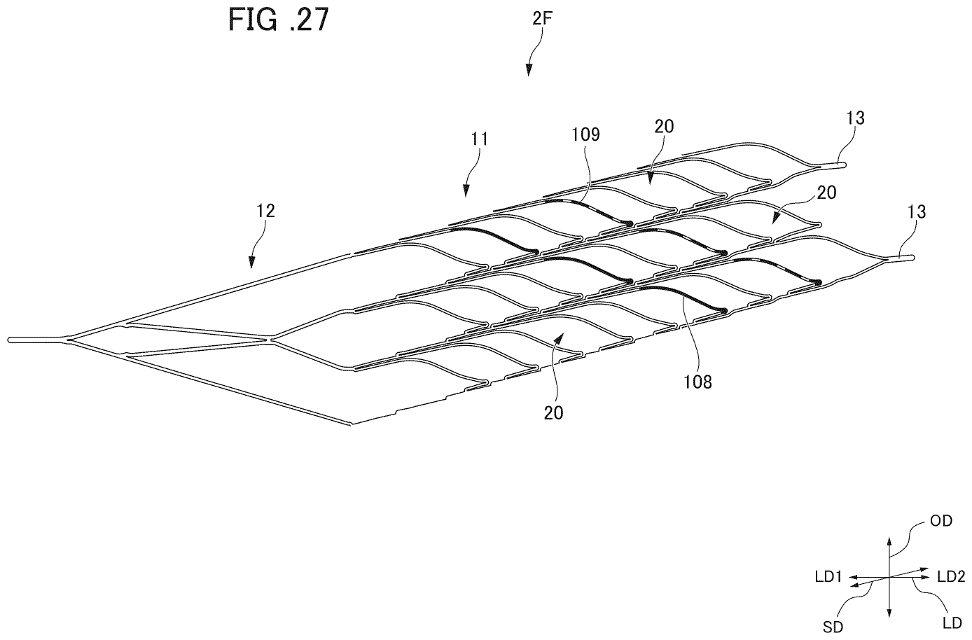

FIG. 27 is an expanded view showing a locking stent 2F according to a sixth embodiment expanded onto a plane virtually. A distal stabilizer 1 according to the sixth embodiment is similar to a distal stabilizer 1 of the second embodiment, except for differences in a shape and location where a radiographically visible part 108 or 109 is provided. Consequently, the same reference symbols are attached to portions functioning similarly to the aforementioned second embodiment, and otherwise redundant descriptions will be omitted as appropriate.

In the locking stent 2F of the sixth embodiment, radiographically visible parts 108 (or radiographically visible parts 109) are provided to struts extending in a direction intersecting an arrangement direction SD. FIG. 27 shows both a radiographically visible part 108 provided continuously along a strut, and radiographically visible parts 109 provided discontinuously like a broken-line along a strut. FIG. 27 illustrates the radiographically visible parts 108 and 109 mixed for explanation. And a radiographically visible part 108 may replace a radiographically visible part 109, and vice versa. In addition, the radiographically visible parts 108 (or the radiographically visible parts 109) may be provided in a plurality of rows as shown in FIG. 27, or may be in one row. In addition, the radiographically visible parts 108 (or the radiographically visible parts 109) may be spaced as shown in FIG. 27 in the arrangement direction SD, or may be aligned continuously.

Since the locking stent 2F of the sixth embodiment is provided with the radiographically visible parts 108 (or the radiographically visible parts 109) in rows aligned in the arrangement direction SD, the diametrically expanded state of the locking stent 2F can be visibly recognized in X-ray observation. In addition, if providing the radiographically visible parts 108 (or the radiographically visible parts 109) in a plurality of rows, the diametrically expanded state for every position in an axial direction LD can be grasped since the radiographically visible parts 108 (or the radiographically visible parts 109) are also arranged spirally, when viewed in the axial direction LD, the diametrically expanded state for every position in the axial direction LD can be grasped.

### Modified Example

The present invention is not limited to the embodiments described above, and various modifications and changes are possible within the scope of the present disclosure.

For example, in a form partially providing the radiographically visible parts of the second embodiment to sixth embodiment, the locking stent having a radiographically visible part may be realized by mounting a marker member having radiopacity to the strut as the radiographically visible part. As a specific form of the marker member, for example, “mechanically fastened marker,” “coil marker,” “radiopaque wire marker,” “radiopaque strut” or the like can be exemplified. It should be noted that the above exemplified forms and expressions thereof are exemplified by categorizing and naming for convenience by the present applicant, and the forms of various conventionally known markers may be used. Mechanically fastened marker is a form fixing a marker member of a ring shape, substantially cylindrical shape or the like that is highly radiopaque by riveting to a strut or the like. Coil marker is a form winding a marker, which is manufactured through forming a wire-like member made of a highly radiopaque material into a coil shape, around a strut or the like to fix with soldering, bonding or the like. Radiopaque wire marker is a form running along or winding around a strut or the like a wire having high radiopacity as a marker, and fixing end portions of the wire to the strut with soldering, bonding or the like. The radiopaque wire marker is a form similar to the aforementioned coil marker; however, it is classified separately from the coil marker since it is not wound so densely to be called a coil. In addition, in the case of the marker member configured as a linear member as described above, for example, it is possible to use a wire of a composite material having a core material, which is made of the aforementioned metal material, coaxially at the center of a wire made of nickel titanium.

It should be noted that, although the respective embodiments and modifications can be employed by combining as appropriate, detailed descriptions thereof are omitted. In addition, the present disclosure is not to be limited by the respective embodiments described above.

### EXPLANATION OF REFERENCE NUMERALS

1 distal stabilizer
2~2F locking stent
3 delivery wire
3f distal end
5 first catheter
6 second catheter
10 delivery system
11 cylindrical portion
12 converging portion
13 distal marker
14 proximal marker
20 cell
21 open cell portion
22a~22d strut
23 free convex end
24 closed cell portion
25 closed convex end
26 apex
101~109 radiographically visible part

1. A distal stabilizer designed for catheter delivery within a biological lumen, the distal stabilizer comprising:
a linear delivery member; and
an anchor device including a cylindrical portion that extends from a distal end of the linear delivery member and is locked to an inner wall of the biological lumen, wherein
the cylindrical portion has a structure in which a plurality of cells, each of which has a shape surrounded by wire-shaped members, are arranged, and
at least a portion of an area in which the cells of the cylindrical portion are provided includes a radiographically visible part.

2. The distal stabilizer according to claim 1, wherein
the radiographically visible part is provided at a part of the cylindrical portion to make a diametrically expanded state thereof visible.

3. The distal stabilizer according to claim wherein
at least one of the cells includes an open cell portion having at least one free convex end, and
the radiographically visible part is provided to at least a wire-shaped member forming the free convex end.

4. An imaging method for acquiring an image of the distal stabilizer according to claim 1 within a biological lumen, the method comprising:
a first step of imaging the radiographically visible part of the distal stabilizer delivered together with the linear delivery member into the biological lumen; and
a second step of imaging the radiographically visible part after moving the cylindrical portion to a position further on a distal side of the biological lumen than a position of the cylindrical portion delivered in the first step.

5. An imaging method for acquiring an image of the distal stabilizer according to claim 1 within a biological lumen, the method comprising:
a first step of imaging the radiographically visible part of the distal stabilizer delivered together with the linear delivery member into the biological lumen, prior to an operation of drawing the linear delivery member; and
a second step of imaging the radiographically visible part after or during the operation of drawing the linear delivery member.

6. An imaging method for acquiring an image of the distal stabilizer according to claim 1 within a biological lumen, the method comprising:
a first step of imaging the radiographically visible part of the distal stabilizer delivered together with the linear delivery member into the biological lumen; and
a second step of imaging the radiographically visible part after the cylindrical portion has shifted from a position thereof within the biological lumen at a time of imaging in the first step.
